(19) United States
(12) Patent Application Publication
(10) Pub. No.: US 2013/0052284 A1
(43) Pub. Date: Feb. 28, 2013

(54) PALM KERNEL OIL-BASED INTRANASAL COMPOSITION AND USE

(71) Applicants: Carolyn J. Harrington, Pittsford, NY (US); Robert A. Harrington, Pittsford, NY (US)

(72) Inventors: Carolyn J. Harrington, Pittsford, NY (US); Robert A. Harrington, Pittsford, NY (US)

(21) Appl. No.: 13/596,688

(22) Filed: Aug. 28, 2012

Related U.S. Application Data
(60) Provisional application No. 61/575,806, filed on Aug. 29, 2011.

(51) Int. Cl. A61K 36/889; A61P 27/00; A61P 29/00; A61P 31/00

(52) U.S. Cl. 424/727

(57) ABSTRACT

The present invention relates to the use of palm kernel oil as an active agent in natural, health-promoting products, particularly for intranasal use.

### CROSS REFERENCE TO RELATED APPLICATIONS

Priority is claimed to: U.S. Provisional Patent Application Ser. No. 61/575,806 by C. Harrington et al., entitled “PALM KERNEL OIL-BASED INTRANASAL COMPOSITION AND USE”, filed on Aug. 29, 2011, the disclosure of which is incorporated herein by reference.

### FIELD OF THE INVENTION

The present invention relates to the use of palm kernel oil as an active agent in natural, health-promoting products, particularly for intranasal use.

### BACKGROUND OF THE INVENTION

Humanity suffers from innumerable ills, among them those of respiratory origin. Increases in world population, lack of proper diet, and rapid travel between distant regions and high concentration of individuals in confined areas, where there is poor air quality, have resulted in an increase in the number of people suffering from respiratory ailments.

The onset of respiratory difficulties is primarily a result of inhalation of airborne material through the nose and mouth. The oral cavity is better equipped to handle airborne materials, such as pathogens, before they can enter the body. Saliva in the mouth captures many airborne materials and stomach acids are highly effective in handling foreign materials before they can enter the body. The nasal passages, on the other hand, are less effective in trapping and killing microorganisms. Airborne materials inhaled through the nose usually enter the lungs and potentially enter the blood stream.

Allergy refers to any condition of the body mounting an attack on a specific foreign substance. People can experience allergic reactions to foods, chemicals, plants, animals and a variety of airborne substances. The symptoms of allergic diseases, such as allergic rhinitis (hay fever), allergic dermatitis, and allergic asthma, can be caused by a variety of allergens, such as grasses, trees, weeds, animal dander, insects, molds, drugs, dust mites, pollen, dust, airborne pollution, and chemicals. The typical symptoms of allergic rhinitis include sneezing, itchy nose, nasal congestion or inflammation, runny nose, and postnasal drip, often accompanied by watery and itchy eyes.

The world is full of microorganisms, and so are people's bodies—in and on the skin, in the gut, and in other orifices. Friendly bacteria are vital to proper development of the immune system, to the protection against microorganisms that could cause disease, and to the digestion and absorption of food and nutrients. Each person's mix of bacteria varies. The balance between a person and the microorganisms in his body, and among the microorganisms themselves, can be crucial to the person's health and well-being. A healthy system with balanced microflora can boost the immune system, keeping pathogenic diseases in check. Unfriendly microorganisms can also upset the balance, triggering the body's immuno-response to many allergens.

Various methods have been utilized to deal with nasal-related conditions, particularly congestion and inflammation. Pharmaceutical agents for infections and allergic diseases have been developed and used. These agents may be provided in the form of sprays, vapor rubs, moisturizers, pills, capsules, liquids, etc. Many such pharmaceutical agents have adverse effects and may be addicting.

### PROBLEM TO BE SOLVED

As a result, there remains a need for antimicrobial and anti-allergic agents derived from natural products, where long-term administration is possible, safety is high, no adverse reaction takes place, and which enhance the inherent protective mechanisms in the nose. In short, there remains a need for a healthier option to combat nasal inflammation and congestion with fewer side effects.

The present invention describes the use of palm kernel oil for intranasal application to promote good health and for treatment and/or prevention of disease and other adverse conditions, particularly nasal inflammation and congestion.

### SUMMARY OF THE INVENTION

The present invention relates to a natural composition comprising palm kernel oil applied to the intranasal cavity. The present invention also relates to a natural composition comprising palm kernel oil, cayenne pepper, lemon balm, and grapefruit seed extract applied to the intranasal cavity. The present invention also relates to a method of providing a healthy intranasal space comprising applying a small amount of an intranasal composition to the tip of a cotton swab, placing the thumb and index finger on the swab stem, placing the cotton tip of the swab just inside of the nostril opening, and using a gentle motion, applying the composition to the surface of the nasal cavity, wherein said intranasal composition comprises palm kernel oil, as well as a method of balancing intranasal microflora comprising applying an intranasal composition to the surface of the nasal cavity, wherein said intranasal composition comprises palm kernel oil.

### ADVANTAGEOUS EFFECT OF THE INVENTION

The present invention includes several advantages, not all of which are incorporated in a single embodiment. The present compositions promote a healthy nose and nasal cavity. The disclosed compositions provide relief from unhealthy nasal conditions, especially those related to unbalanced microbial growth, which can lead to congestion and inflammation. The compositions may have antimicrobial properties, particularly antibacterial and antiviral properties.

### DETAILED DESCRIPTION OF THE INVENTION

The present invention relates to a composition comprising palm kernel oil for topical administration. More specifically, the present disclosure provides natural compositions comprising palm kernel oil as an active ingredient for promoting a healthy intranasal space. The present inventive composition may contain additional active ingredients having natural, homeopathic, medicinal, pharmaceutical, and/or cosmetic properties. In addition, the present invention relates to methods of using these compositions to promote good health of the intranasal space as well as alleviate unhealthy conditions and any related symptoms in the intranasal space.

As used herein, the terms “active ingredient” and “active agent” refer to ingredients that produce a natural, homeopathic, medicinal, pharmaceutical or cosmetic effect.

The term “nasal cavity,” refers to all aspects of the nasal cavity, including the nostrils, nasal/mucus membrane, cilia, and sinuses.

The term “intranasal space” refers to the special cavity inside the nose.

Medium-chain triglycerides (MCTs) are medium-chain (6 to 12 carbons) fatty acid esters of glycerol.

Palm kernel oil is recognized as a valuable natural product that promotes health in a variety of ways. Palm kernel oil is useful as a nutritional component, skin emollient and is also reputed to have antimicrobial properties, antifungal and antiviral properties. Palm kernel oils are edible plant oils derived from the kernel (seed) of the oil palm. Palm kernel oil is a highly saturated vegetable fat.

Palm kernel oil has a fatty acid profile that is almost identical to that of coconut oil. Since palm kernel oil and coconut oil have very high contents of lauric acid, they are referred to as lauric oils. Palm kernel oil is composed of fatty acids, esterified with glycerol just like any ordinary fat. It is high in saturated fatty acids, about 80%. The approximate concentration of fatty acids (FAs) in palm kernel oil is lauric (saturated C12) 48.2%, myristic (saturated C14) 16.2%, palmitic (saturated C16) 8.4%, capric (saturated C10) 3.4%, caprylic (saturated C8) 3.3%, stearic (saturated C18) 2.5%, oleic (monounsaturated C18) 15.3%, linoleic (polyunsaturated C18) 2.3%, other/unknown 0.4%.

Palm kernel oil is present as the primary active ingredient in the present invention. Palm kernel oil may be present in the compositions in an amount ranging from 0.1-100% by weight of the composition. Preferably, the palm kernel oil comprises a fractionated palm kernel oil. Preferably, the palm kernel oil comprises a fractionated palm kernel oil, where the long and short chain fatty acids are removed from the composition, leaving behind the medium chain fatty acids.

In another embodiment, the invention relates to the use of medium-chain triglycerides, which contain fatty acids called medium-chain fatty acids, from other sources, instead of the palm kernel oil specifically. The names of the medium-chain fatty acids found in MCTs are caproic acid (C6), caprylic acid (C8), capric acid (C10) and lauric acid (C12). MCTs are composed of a glycerol backbone and three of these fatty acids.

The compositions may also comprise at least one other ingredient having natural, homeopathic, medicinal, pharmaceutical, and/or cosmetic properties. Most preferably, the other ingredients are all natural ingredients, to produce a completely natural composition. Any additional active agents may be present in an amount ranging from 0.1-99.9% by weight of the composition, for example from 0.1-50% by weight, 0.1-25% by weight, 0.1-10% by weight, 0.1-5% by weight, or even 0.1-0.9% by weight of the composition.

The biggest chemical barrier to infectious organisms is the acid layer on the skin. Healthy skin has a pH of 5, making it slightly acidic. Our sweat and body oils promote this acidic environment. Harmless bacteria can tolerate the acid and live on the skin, but troublesome bacteria can't thrive and their numbers are few.

The oil our bodies produce is called sebum. Sebum is created by sebaceous oil glands located at the root of every hair. This oil is very important to skin health. Sebum contains medium chain fatty acids, in the form of medium chain triglycerides, that can be released to fight harmful organisms. At least one type of bacterium normally present on the skin feeds on the sebum, breaking down the triglycerides into free fatty acids.

The bacteria feed on the glycerol part of the triglyceride, freeing the fatty acids from the glycerol unit. Medium chain fatty acids have no immediate antimicrobial properties, when bound to glycerol units, as they are in coconut and palm kernel oils. Freed fatty acids, however, have antimicrobial properties.

The combination of the slightly acidic skin pH and the medium chain fatty acids provides a protective chemical layer on the skin that prevents infection from disease-causing organisms. Due primarily to the action of bacteria, the oil on the surface of the hair and skin is composed of between 40 to 60 percent free fatty acids. The medium chain fatty acids in the sebum provide the protective layer on the skin that kills harmful germs. When medium chain triglyceride oils derived from coconut oil or palm kernel oil is put on the skin, it doesn't have immediate antimicrobial action. However, when bacteria on the skin turn these triglycerides into free fatty acids, the result is an increase in the number of antimicrobial fatty acids on the skin, resulting in protection from infection.

In a preferred embodiment, the composition includes cayenne pepper, lemon balm, grapefruit seed extract, and candelilla wax.

Cayenne pepper is a red, hot chili pepper mostly known for its use in spicy dishes, however, there are also a great many associated health benefits. Used in the west since the 17th century, it has been found to treat such things as fever, cold, diarrhea; it relieves constipation, headache, and sinus congestion. Applied externally (to tired feet, sore muscles, strains and sprains) it acts as a pain reliever through a little known numbing effect. The active ingredient, capsaicin, from which cayenne's genus name, capsicum, is derived, is responsible for both cayenne's heat as well as its medicinal function. One of the most impressive properties of cayenne pepper is its quick absorption and consequent fast action. A pinch in each nostril starts working immediately to remove excess mucous and relieve sinus congestion. A dab of capsaicin extract placed in the nose has been said to relieve headaches.

Lemon balm is used medicinally as an herbal tea, or in extract form. It is claimed to have antibacterial and antiviral properties. It is also used as an anxiolytic, mild sedative or calming agent. Lemon balm and preparations thereof also have shown to improve mood and mental performance. The extract of lemon balm was also found to have exceptionally high antioxidant activity. Lemon balm contains eugenol, which kills bacteria and has been shown to calm muscles and numb tissues and is an antispasmodic. It also contains tannins that contribute to its antiviral effects, as well as terpenes that add to its soothing effects.

Grapefruit seed extract has been used for its antioxidant properties. Analysis shows the constituents of the seed extract and pulp are flavonoids, ascorbic acid (commonly known as vitamin C), tocopherols, citric acid, limonoids, sterols, and minerals.

The compositions of the present invention may also contain other pharmaceutically and/or cosmetically acceptable ingredients, including, but not limited to, emollients, moisturizers, conditioners, antioxidants, fillers, dyes and other coloring agents, preservatives, emulsifying agents, surfactants, stabilizers, thickeners, and other materials known in the art.

A variety of additional ingredients can be incorporated into the composition of the present invention. Non-limiting examples of these additional ingredients include vitamins and derivatives thereof (e.g. tocopherol, panthenol), thickening agents, saturated and/or unsaturated alkyl alpha hydroxy acids, resins, gums, waxes (both naturally occurring and synthetic, for example, beeswax), polymers, abrasive scrub particles, preservatives, skin penetration aids, skin bleaching (or lightening) agents, chelators and sequestrants, and aesthetic components such as essential oils, skin sensates, astringents, skin soothing agents, skin healing agents. Non-limiting examples of these aesthetic components include aloe vera, pantothenic acid and its derivatives, clove oil, menthol, camphor, eucalyptus oil, eugenol, menthyl lactate, witch hazel distillate, allantoin, bisabalol, and dipotassium glycyrrhizinate.

Examples of suitable emollients include, but are not limited to, volatile and non-volatile silicone oils (e.g., dimethicone, cyclomethicone, dimethiconol, and the like), highly branched hydrocarbons, and mixtures thereof, sunflower (Helianthus annuus) oil, canola (Brassica napus/campestris) oil, sweet almond (Prunus amygdalus dulcis) oil, sesame (Sesamum indicum) oil, and macadamia (Macadamia ternifolia) nut oil. The emollients can typically comprise in total from about 0.1 percent to about 25 percent, more preferably from about 0.5 percent to about 10 percent, and most preferably from about 0.5 percent to about 5 percent by weight of the composition.

The composition may also comprise a suitable gelling agent, thickening or other texture modification ingredients. A preferred texturizing component is candelilla wax. Useful gelling or thickening agents may include, but are not limited to, cellulose esters such as hydroxypropyl cellulose, hydroxyethyl cellulose, polyvinylpyrrolidone, carboxyvinyl polymer to thicken the composition to a desired gel consistency.

The compositions are preferably in a form suitable for topical application, particularly as an ointment for application to the nasal cavity.

Although the preferred embodiment is an ointment for intranasal application, other forms may be useful, such as emulsions, suspensions, creams, lotions, gels, liquids, solids, and sprays.

The present includes methods of using these compositions to promote good health and/or for treatment and/or prevention of disease. In use, the composition is applied to the nasal cavity as follows:
a. apply a small amount to the tip of a cotton swab or a tissue;
b. place the thumb and index finger on the swab stem;
c. carefully place the cotton tip of the swab just inside of the nostril opening;
d. using a gentle motion, make 3 or 4 circles to fully apply the composition to the nasal cavity, being careful not to insert more than half way into your nasal cavity;
e. discard the swab; and
f. repeat these steps for the other nostril.

The present method is not limited to a swab or tissue-type applicator. Other applicators which may be safely inserted into the nasal cavity may be used, for example, a fingertip.

The invention has been described in detail with particular reference to certain preferred embodiments thereof, but it will be understood that variations and modifications can be effected within the spirit and scope of the invention.

1. A composition comprising palm kernel oil applied to the intranasal cavity.

2. The composition of claim 1 wherein said palm kernel oil is fractionated palm kernel oil.

3. The composition of claim 1 wherein said composition provides antimicrobial properties.

4. The composition of claim 1 wherein said composition provides decongestant or anti-inflammatory properties.

5. The composition of claim 1 wherein said composition provides prebiotic or probiotic properties through balanced microflora.

6. The composition of claim 1 further comprising at least one of cayenne pepper, lemon balm, grapefruit seed extract, and candelilla wax.

7. A composition comprising palm kernel oil, cayenne pepper, lemon balm, and grapefruit seed extract, applied to the intranasal cavity.

8. The composition of claim 7 further comprising candelilla wax.

9. A method of providing a healthy intranasal space comprising:
a. applying a small amount of an intranasal composition to the tip of a cotton swab;
b. placing the thumb and index finger on the swab stem;
c. placing the cotton tip of the swab just inside of the nostril opening; and
d. using a gentle motion, applying the composition to the surface of the nasal cavity, wherein said intranasal composition comprises palm kernel oil.

10. A method of balancing intranasal microflora comprising:
a. applying an intranasal composition to the surface of the nasal cavity, wherein said intranasal composition comprises palm kernel oil.
